(19) United States
(12) Patent Application Publication
(10) Pub. No.: US 2025/0378558 A1
(43) Pub. Date: Dec. 11, 2025

(54) AGGREGATED REPRESENTATION OF A VESSEL STRUCTURE

(71) Applicant: Siemens Healthineers AG, Forchheim (DE)

(72) Inventors: Dominik NEUMANN, Erlangen (DE); Alexandru TURCEA, Busteni (RO)

(73) Assignee: Siemens Healthineers AG, Forchheim (DE)

(21) Appl. No.: 19/231,852

(22) Filed: Jun. 9, 2025

(30) Foreign Application Priority Data
Jun. 10, 2024 (EP) 24465529.6

(51) Int. Cl. G06T 7/00; G06T 7/11; G06T 7/174; G16H 30/40

(52) U.S. Cl. CPC G06T 7/0012; G06T 7/11; G06T 7/174; G16H 30/40; G06T 2207/30101

(57) ABSTRACT

Graph representations of a vessel structure are received, each corresponding to a medical image and including a plurality of nodes and respective spatial coordinates for each node. The plurality of nodes are arranged according to at least one segment. For each respective segment of a first graph representation, it is determined whether a second graph representation of the plurality of graph representations includes a segment corresponding to the respective segment of the first graph representation and, if it is found that this is not the case, the second graph representation is augmented by adding an additional segment corresponding to the respective segment of the first graph representation. An aggregated representation of the vessel structure is generated depending on the augmented second graph representation.

### CROSS-REFERENCE TO RELATED APPLICATION(S)

The present application claims priority under 35 U.S.C. § 119 to European Patent Application No. 24465529.6, filed Jun. 10, 2024, the entire contents of which are incorporated herein by reference.

### FIELD

One or more example embodiments of the present invention are directed to a computer-implemented method for generating at least one aggregated representation of a vessel structure, to a method for generating at least one aggregated representation of a vessel structure, to a data processing system for carrying out said computer-implemented method, to a medical imaging system comprising such a data processing system, and to a corresponding non-transitory computer program product.

### BACKGROUND

Medical imaging of vessel structures, also denoted as angiography, is widely used for assisting at medical interventions. In particular, invasive coronary X-Ray angiography is the method of choice for the assessment of coronary arteries and the diagnosis of coronary artery disease, CAD. Software tools enhanced by artificial intelligence, AI, components are slowly finding their entry into the cath labs to support interventional cardiologists in their decision making, potentially leading to improved efficiency, diagnostic accuracy, and reproducibility.

Over the last years, several modules for different tasks such as vessel detection, vessel segmentation, branch labeling, computation of the fractional flow reserve, FFR, or another hemodynamic characteristics, and so forth have been developed, many of them based on AI. Some of these modules have the advantage that their analysis is not isolated to a single frame within an angiography sequence, but instead it aggregates information across multiple frames, for example over a full heart cycle, which aims to get a better understanding of the true state of the anatomy and physiology of the patient's coronaries.

However, aggregation of information extracted from multiple frames poses major challenges. For example, it may happen that not all portions of the vessel structure can be extracted from each individual frame, for example due to the motion of the patient, the cardiac motion, changing angulation of the imaging device and so forth, which leads to inconsistencies in the representation of the vessel structure. This may reduce the reliability and/or accuracy of the subsequent tasks' results. It would therefore be desirable to have a more universal representation of a vessel structure over several frames of medical images.

### SUMMARY

It is an objective of one or more example embodiments of the present invention to provide a more consistent representation of a vessel structure over several frames of medical images.

At least this objective is achieved by the subject matter of the independent claim. Further implementations and preferred embodiments are subject matter of the dependent claims.

One or more example embodiments of the present invention are based on the idea to generate at least one aggregated representation of a vessel structure based on respective graph representations of the same vessel structure depicted in medical images by attaching segments of a first graph representation to a second graph representation, in case the second graph representation does not comprise a respective segment already.

According to an aspect of embodiments of the present invention, a computer-implemented method for generating at least one aggregated representation of a vessel structure is provided. Therein, respective graph representations of the vessel structure are received. Therein, each graph representation of the received graph representations corresponds to a respective medical image of a sequence of consecutive medical images, each of the medical images depicting the vessel structure. Each of the graph representations comprises a plurality of nodes, in particular consists of the plurality of nodes and edges connecting the nodes, and respective spatial coordinates for each node of the respective plurality of nodes. Therein, the plurality of nodes is arranged according to at least one segment of the respective graph representation. For each first segment of the plurality of segments of a first graph representation of the plurality of graph representations, the following steps i) and ii) are carried out. Therein, segments of the plurality of segments of a first graph representation of the plurality of graph representations are denoted as first segments and segments of the plurality of segments of a second graph representation of the plurality of graph representations are denoted as second segments. It is noted that the plurality of graph representations is not necessarily ordered in a specific way and, in particular, the first graph representation is not necessarily an initial graph representation according to such an order and, in particular, the second graph representation does not necessarily follow the first graph representation according to such an order.

In step i), it is determined whether the second graph representation of the plurality of graph representations comprises a second segment corresponding to the respective first segment of the first graph representation, in particular depending on the spatial coordinates of the nodes of the first segment and the nodes of the second graph representation. In step ii), the second graph representation is augmented if it is found that the second graph representation does not comprise a second segment corresponding to the respective first segment of the first graph representation in step i). Therein, augmenting the second graph representation comprises adding an additional second segment corresponding to the respective first segment of the first graph representation to the second graph representation, for example depending on the spatial coordinates of the nodes of the first segment and the nodes of the second graph representation. After steps i) and ii) have been carried out for all first segments of the first graph representation, the at least one aggregated representation is generated depending on the augmented second graph representation.

Unless stated otherwise, all steps of the computer-implemented method may be performed by a data processing system, which comprises at least one data processing device. In particular, the at least one data processing device is configured or adapted to perform the steps of the computer-implemented method. For this purpose, the at least one data processing device may for example store a computer program comprising instructions which, when executed by the at least one data processing device, cause the at least one data processing device to execute the computer-implemented method. The expressions “data processing system” and “at least one data processing device” may be used interchangeably, here and in the following. This holds also for respective expressions derived therefrom.

In case the at least one data processing device comprises two or more data processing devices, certain steps carried out by the at least one data processing device may also be understood such that different data processing devices carry out different steps or different parts of a step. In particular, it is not required that each data processing device carries out the steps completely. In other words, carrying out the steps may be distributed amongst the two or more data processing devices.

From each implementation of the computer-implemented method, a respective implementation of a method for generating at least one aggregated representation of a vessel structure, which is not purely computer-implemented, is obtained by including respective steps of generating the sequence of consecutive medical images, in particular by the imaging device.

The imaging device may be any medical imaging device, which is capable of generating respective medical images depicting the vessel structure, which is a vessel structure of a patient, for example a human or animal, for example after a contrast agent has been applied. In particular, the imaging device may be an X-ray imaging device, in particular an X-ray-based angiography device, for example a C-arm device. The medical images may therefore be two-dimensional X-ray projection images. The medical images may, however, also be three-dimensional X-ray images, for example tomosynthesis reconstructions. The imaging device may also be a computed tomography, CT-, device, for example a CT-device for four-dimensional CT also denoted as time resolved CT, and the medical images may be three-dimensional CT-reconstructions. It is also possible that the imaging device is a magnetic resonance imaging, MRI-, device, in particular an MRI device for time resolved MRI, for example time resolved angiography.

In particular, each of the medical images depicts the vessel structure at a different time. In particular, the sequence of consecutive medical images corresponds to a sequence of consecutive frames and each graph representation corresponds to one of the frames. The sequence of consecutive medical images may consist of two or more medical images. Consequently, the sequence of consecutive frames may consist of two or more frames. Thus, the graph representations may consist of the first graph representation and the second graph representation or may comprise more than said two graph representations. In particular, there is a one-to-one correspondence between the graph representations and the sequence of medical images or the sequence of frames, respectively. However, the graph representations do not necessarily need to be processed in an ordered manner which is identical to the ordering of the medical images or frames, respectively.

It is also not necessary that the graph representations are received all at once, while this is possible in some implementations. In particular, all the graph representations may be received after the sequence of medical images has been generated. However, it is also possible that the graph representations are received one after another when the respective medical image has been generated.

It is noted that the augmented second graph representation may be augmented exactly once or multiple times depending on how many first segments of the first graph representation do not have respective counterparts in the second graph representation. It is also possible that the second graph representation comprises respective second segments for all first segments of the first graph representation from the beginning. In this case, no additional segment is added to the second graph representation. Nevertheless, the augmented second graph representation is also referred to in such cases.

The at least one aggregated representation may consist of the augmented second graph representation. In particular, the augmented second graph representation is an aggregated graph representation of the original first graph representation and second graph representation. It is, however, also possible that the augmented second graph representation is further processed to generate the at least one aggregated representation.

For example, the steps i) and ii) may be carried out in the same way as described also for a third graph representation and a fourth graph representation of the received graph representations, which differ from the first graph representation and the second graph representation. In this case, for example an augmented fourth graph representation is generated and the at least one aggregated representation is generated depending on the augmented second graph representation and the augmented fourth graph representation.

It is also possible that the steps i) and ii) are carried out again in the same way as described, wherein the augmented second graph representation takes the place of the first graph representation and a third graph representation of the received graph representations takes the place of the second graph representation. In this case, for example an augmented third graph representation is generated and the at least one aggregated representation is generated depending on the augmented second graph representation and the augmented third graph representation. This may, for example, be repeated analogously until all graph representations of the received graph representations are considered accordingly. It is also possible to group the graph representations into different groups and carry out the described procedures for each group individually.

It is, in particular, possible that, as a final result, a single aggregated representation is obtained. Said single aggregate representation would then have been generated depending on the augmented second graph representation.

The expression graph representation can, in particular, be understood as a graph in the context of graph theory. The nodes of a graph representation have respective attributes, namely at least the respective spatial coordinates, which may also be denoted as image coordinates. The spatial coordinates of the nodes of a given graph representation are, for example, given in a respective reference coordinate system of the corresponding graph or the corresponding medical image. Since the graph representation comprises the respective spatial coordinates for each node, the data processing system is able to determine whether for a given first segment the second graph representation comprises a corresponding second segment and, if this is not the case, augment the second graph representation as described. To check whether the second graph representation comprises a second segment corresponding to the respective first segment of the first graph representation, for example a coordinate-based distance metric between all nodes of the first segment and their closest counterparts in the second graph representation may, for example, be evaluated.

It is noted that the decision whether or not the second graph representation comprises a second segment corresponding to the respective first segment of the first graph representation can be based on different thresholds or tolerances depending on the actual embodiment of the computer-implemented method. This means that, in some implementations, it may be considered that the second graph representation does not comprise a second segment corresponding to the respective first segment, even though the second graph representation does comprise a part of such a second segment, which is, however, smaller or contains fewer nodes, respectively, than the respective first segment to an extent exceeding a predefined tolerance.

The extraction of the vessel structure from the respective medical image and the generation of the graph representations based on the medical images are not necessarily steps, which are comprised by the computer-implemented method, according to one or more example embodiments of the present invention, and can be carried out using known techniques. However, in some embodiments, the steps of extracting the vessel structure from the respective medical image and generating the graph representations based on the medical images are part of the computer-implemented method.

In several embodiments, each of the graph representations is given by a graph representation 11, for example a rooted graph representation 11.

In particular, each node of one of the graph representations has either one parent node or no parent node. In each graph representation, exactly one node without a parent node exists and this node is denoted as root node. A node can have exactly one child node or more than one child nodes or no child node at all. Nodes, which have no child nodes are denoted as leaf nodes and nodes with more than one child node are denoted as multifurcation node, in particular bifurcation nodes for exactly two child nodes, trifurcation nodes for exactly three child nodes, and so forth.

Each graph representation comprises one or more segments, which may also be denoted as branches. Each segment comprises a respective subset of the nodes of the respective graph representation. In particular, a segment may be understood such that all nodes of the respective segment except an initial node of the segment have a parent node in the same segment and all nodes of the respective segment except a final node of the segment have exactly one child node in the same segment. Consequently, the initial node is either the root node or its parent node is part of another segment of the same graph representation. Analogously, the final node is either a leaf node or it has two or more child nodes in other segments of the same graph representation. In other words, a segment may, for example, be understood as a subset of pairwise connected nodes of a graph representation without multifurcations.

When assessing a vessel structure, for example in order to determine certain hemodynamic characteristics, in particular by a trained machine learning model, MLM, different phenomena may cause the individual medical images to not all show the same portions of the vessel structure and/or to not all show the same portions of the vessel structure completely. These phenomena may, for example, include body movement of the patient, cardiac or respiratory motion, the dynamics of the contrast agent bolus and so forth. These phenomena may further be accentuated in case the imaging settings or protocol for generating the medical images are not chosen in an optimal way, in particular in case of two-dimensional imaging. The respective algorithm for extracting the vessel structure and/or generating the graph representations based on the medical images may therefore not generate the same segments for all graph representations, even though they all represent the same vessel structure.

The computer-implemented method uses a graph aggregation approach achieving a robust aggregation of potentially inconsistent graph representations of the vessel structure, which were individually extracted from multiple frames of the same angiography sequence, in particular the same underlying anatomy and the same patient but at different time points, for example at different heart phases, different image quality and hence different quality of extracted graph representations. The at least one aggregated representation overcomes these inconsistencies at least partially. Using the at least one aggregated representation as a basis or as auxiliary information for downstream tasks, in particular for characterizing the hemodynamics in the vessel structure, improves the quality of the results of said downstream tasks and, consequently the quality of clinical decisions made based on said results and/or the chances of success of a medical intervention, which uses the results of said downstream tasks.

According to several implementations of a first embodiment, the graph representations, in particular all received graph representations, are ordered into a single ordered sequence of graph representations. The total number of graph representations of the ordered sequence is N. The graph representations may be indexed by integer numbers i from 1 to N, in other words i∈[1,N]∩N. N−1 iterations are carried out, wherein the N−1 iterations start with i=1 and end with i=N−1 and after each iteration, i is incremented by 1 until i=N−1. For each of the N−1 iterations and for each segment of the i-th graph representation of the ordered sequence,
i) it is determined whether the (i+1)-th graph representation of the ordered sequence comprises a segment corresponding to the respective segment of the i-th graph representation of the ordered sequence, and
ii) if it is found that the (i+1)-th graph representation does not comprise a segment corresponding to the respective segment of the i-th graph representation, the (i+1)-th graph representation is augmented by adding an additional segment corresponding to the respective segment of the i-th graph representation to the (i+1)-th graph representation.

The at least one aggregated representation is generated depending on, for example comprising or consisting of, the augmented N-th graph representation of the ordered sequence, in particular when all of the N−1 iterations have been carried out.

In other words, the steps explained above for the first graph representation and the second graph representation are carried out iteratively for each pair of consecutive graph representations in the ordered sequence. More specifically, in a given iteration i, the i-th graph representation corresponds to the first graph representation in the earlier explanations above and the (i+1)-th graph representation corresponds to the second graph representation in the earlier explanations above. It is noted that the first graph representation and the second graph representation are also part of the ordered sequence of graph representations. Consequently, the N−1 iterations as described also comprise one iteration, where the i-th graph representation is the first graph representation and the (i+1)-th graph representation is the second graph representation. The described steps are, in particular, not carried out twice for the identical first and second graph representation.

It is further noted that the ordering of the ordered sequence of graph representations may match the order ring of the sequence of consecutive medical images, but this is not necessarily the case.

In such embodiments, the augmented N-th graph representation should comprise all segments of all graph representations of the ordered sequence prior to the N−1 iterations. In particular, the augmented N-th graph representation is an aggregated graph representation of all graph representations of the ordered sequence. Therefore, the N-th graph representation is particularly suitable as an aggregated graph representation for the purposes explained above.

The at least one aggregated representation may consist of the augmented N-th graph representation. It is, however, also possible that the augmented N-th graph representation is further processed to generate the at least one aggregated representation.

According to several implementations of the first embodiment, after the N−1 iterations have been carried out, N−1 further iterations are carried out. For the further iterations, the graph representations may be indexed by integer numbers j from 1 to N, in other words j∈[1,N]∩N. The N−1 further iterations start with j=1 and end with j=N−1 and after each iteration, j is incremented by 1 until j=N−1. For each iteration of the N−1 further iterations and for each segment of the (N−j+1)-th graph representation of the ordered sequence,
iii) it is determined whether the (N−j)-th graph representation of the ordered sequence comprises a segment corresponding to the respective segment of the (N−j+1)-th graph representation of the ordered sequence;
iv) if it is found that the (N−j)-th graph representation does not comprise a segment corresponding to the respective segment of the (N-j+1)-th graph representation, the (N−j)-th graph representation is augmented by adding an additional segment corresponding to the respective segment of the (N−j+1)-th graph representation to the (N−j)-th graph representation The at least one aggregated representation is generated depending on, for example comprising or consisting of, the augmented initial graph representation, which is the graph representation with j=1, of the ordered sequence, in particular when all of the N−1 further iterations have been carried out. For example, the at least one aggregated representation may be generated depending on, for example comprising or consisting of, all graph representations of the ordered sequence, after the N−1 further iterations have been carried out.

In other words, after the N−1 iterations have been carried out as described above, the N−1 further iterations are carried out based on the resulting ordered sequence in the opposite order. While the N−1 iterations start with the 1-th and the 2-th graph representation (i=1) and end with the (N−1)-th and N-th graph representation (i=N−1), the N−1 further iterations start with the N-th and the (N−1)-th graph representation (j=1) and end with the 2-th and 1-th graph representation (j=N−1).

In such embodiments, after the N−1 further iterations have been carried out, all graph representations of the ordered sequence should comprise all segments of all graph representations of the ordered sequence prior to the N−1 iterations. Therefore, after the N−1 further iterations have been carried out, the graph representations are particularly suitable as aggregated graph representations for the purposes explained above, in particular for downstream applications, where a graph representation is required for each frame of the sequence of consecutive frames.

According to several implementations of a second embodiment, the graph representations, in particular all received graph representations, are grouped into at least two ordered sequences of graph representations including a first ordered sequence and a second ordered sequence. The total number of graph representations of the first ordered sequence is N1. The graph representations of the first ordered sequence may be indexed by integer numbers i from 1 to N, in other words i∈[1,N1]∩N. N1−1 iterations are carried out, wherein the N1−1 iterations start with i=1 and end with i=N1−1 and after each iteration, i is incremented by 1 until i=N1−1. For each iteration of the N1−1 iterations and for each segment of the i-th graph representation of the first ordered sequence,
i) it is determined whether the (i+i)-th graph representation of the first ordered sequence comprises a segment corresponding to the respective segment of the i-th graph representation of the first ordered sequence, and
ii) if it is found that the (i+i)-th graph representation of the first ordered sequence does not comprise a segment corresponding to the respective segment of the i-th graph representation of the first ordered sequence, the (i+i)-th graph representation of the first ordered sequence is augmented by adding an additional segment corresponding to the respective segment of the i-th graph representation of the first ordered sequence to the (i+i)-th graph representation of the first ordered sequence.

The at least one aggregated representation is generated depending on the augmented N1-th graph representation of the first ordered sequence, in particular when all of the N1−1 iterations have been carried out.

The explanations regarding the N−1 iterations of steps i) and ii) of the first embodiment hold analogously here for the second embodiment. In the implementations according to the second embodiment, however, the steps are not carried out for all of the graph representations one after the other, but only for a subset, namely the first ordered sequence. The other ordered sequences of the at least two ordered sequences, in particular the second ordered sequence, may be treated analogously. An advantage of such implementations is that the iterations may be carried out for the at least two ordered sequences in parallel, which reduces the overall required for the computation.

The at least two ordered sequences may be defined in an arbitrary way. For example, if the at least two ordered sequences consist of K ordered sequences, they may be defined such that each ordered sequences comprises 1/K or approximately 1/K of all the graph representations.

According to several implementations of the second embodiment, the total number of graph representations of the second ordered sequence is N2. The graph representations of the second ordered sequence may be indexed by integer numbers i from 1 to N, in other words i∈[1,N2]A N. N2−1 further iterations are carried out, wherein the N2−1 further iterations start with i=1 and end with i=N1−1 and after each further iteration, i is incremented by 1 until i=N2−1. For each further iteration of the N2-1 further iterations and for each segment of the i-th graph representation of the second ordered sequence,
iii) it is determined whether the (i+1)-th graph representation of the second ordered sequence comprises a segment corresponding to the respective segment of the i-th graph representation of the second ordered sequence, and
iv) if it is found that the (i+1)-th graph representation of the second ordered sequence does not comprise a segment corresponding to the respective segment of the i-th graph representation of the second ordered sequence, the (i+1)-th graph representation of the second ordered sequence is augmented by adding an additional segment corresponding to the respective segment of the i-th graph representation of the second ordered sequence to the (i+1)-th graph representation of the second ordered sequence.

The at least one aggregated representation is generated depending on the augmented N2-th graph representation of the second ordered sequence, in particular when all of the N2-1 iterations have been carried out.

According to several implementations of the second embodiment, the N1−1 iterations, in particular steps i) and ii), and the N2−1 further iterations, in particular steps iii) and iv), are carried out in parallel or partially in parallel.

For example, different data processing devices or computing cores or the like may be used to carry out the N1−1 iterations and the N2−1 iterations, respectively.

According to several implementations of the second embodiment, for each segment of the N1-th graph representation of the first ordered sequence,
v) it is determined whether the N2-th graph representation of the second ordered sequence comprises a segment corresponding to the respective segment of the N1-th graph representation of the first ordered sequence, and
vi) if it is found that the N2-th graph representation of the second ordered sequence does not comprise a segment corresponding to the respective segment of the N1-th graph representation of the first ordered sequence, the N2-th graph representation of the second ordered sequence is augmented by adding an additional segment corresponding to the respective segment of the N1-th graph representation of the first ordered sequence to the N2-th graph of the second ordered sequence.

The at least one aggregated representation is generated depending on the augmented N2-th graph representation of the second ordered sequence.

In such embodiments, the augmented N2-th graph representation should comprise all segments of all graph representations of the first ordered sequence prior to the N1−1 iterations and the second ordered sequence prior to the N2-1 further iterations. Therefore, the N2-th graph representation is particularly suitable as an aggregated graph representation for the purposes explained above.

As explained for the respective implementations of the first embodiment, also in implementations of the second embodiment, additional aggregation steps may be carried out in the reverse order.

According to several implementations of the computer-implemented method, the first graph representation is registered to the second graph representation, in particular depending on the spatial coordinates of the nodes of the first graph representation and the spatial coordinates of the nodes of the second graph representation. A predefined distance metric between the registered respective segment of the first graph representation and the second graph representation is computed, in particular depending on the spatial coordinates of the nodes of the respective segment of the first graph representation after the registration and the spatial coordinates of the nodes of the second graph representation. It is determined whether the second graph representation comprises a segment corresponding to the respective segment of the first graph representation depending on the computed distance metric.

For example, if the computed distance metric is greater than a predefined threshold value, then the segment may be considered missing in the second graph representation. The registration can be a rigid registration or a deformable registration. In particular, registering the first graph representation to the second graph representation comprises finding a transformation, which maps the nodes of the first graph representation to corresponding nodes of the second graph representation, in case the second graph representation comprises such corresponding nodes. The registered segment of the first graph representation is then given by the accordingly transformed segment of the first graph representation.

Via the registration, it is explicitly accounted for a possible movement of the patient or the vessel structure while the medical images have been acquired. Thus, the accuracy of the aggregation is increased.

Instead of said registration approach, it is also possible to apply an accordingly trained MLM to the first graph representation and the second graph representation to identify all segments of the first graph representation which do not have a corresponding counterpart in the second graph representation.

According to several implementations of the computer-implemented method, a deformation field between the first graph representation and the second graph representation is determined, in particular depending on the spatial coordinates of the nodes of the first graph representation and the spatial coordinates of the nodes of the second graph representation. The second graph representation is augmented by adding the additional segment to the second graph representation depending on the deformation field.

The deformation field is, in particular, a transformation which maps each node of the first graph representation into the coordinate system of the second graph representation, such that the resulting transformed first graph representation optimally matches the second graph representation. Finding the deformation field can be considered and solved as an optimization problem using known methods, in particular methods known from deformable image registration techniques.

In principle, it is also possible to augment the second graph representation by adding the additional segment without the deformation field, in particular without any such transformation. This implicitly or explicitly assumes that the graph representations corresponding to different frames have not significantly moved with respect to each other. By adapting the coordinates of the nodes of the additional segment according to the deformation field, the accuracy of the proposed method is therefore increased.

According to a further aspect of one or more example embodiments of the present invention, a method for generating at least one aggregated representation of a vessel structure, which is not purely computer-implemented, is provided. Therein, a sequence of consecutive medical images depicting the vessel structure is generated by an imaging device and a respective graph representation of the vessel structure is generated for each of the sequence of consecutive medical images, for example by the data processing system. A computer-implemented method according to one or more example embodiments of the present invention is carried out based on the graph representations, in particular by the data processing system.

According to several implementations of the method, the sequence of consecutive medical images is generated as a sequence of consecutive two-dimensional images, for example two-dimensional X-ray images.

In such implementations, one or more example embodiments of the present invention are particularly beneficial since, due to the two-dimensional nature of the medical images, the chances that parts of the vessel structure are not consistently depicted in each medical image, for example due to occlusion by other parts of the vessel structure or by other objects, are increased. Consequently, the at least one aggregated representation of the vessel structure is particularly helpful in this case.

According to several implementations, the sequence of consecutive medical images contains medical images from at least two viewing directions to the vessel structure.

Also in such implementations, one or more example embodiments of the present invention are particularly beneficial, since it often cannot be guaranteed that the optimal imaging settings for both viewing directions are chosen by the operator, such that the chances for inconsistency among the medical images is also increased.

According to a further aspect of one or more example embodiments of the present invention, a method for analyzing a hemodynamics characteristics of a vessel structure is provided. Therein, a method or computer-implemented method for generating at least one aggregated representation of the vessel structure, according to one or more example embodiments of the present invention, is carried out. A predefined algorithm for analyzing the hemodynamics characteristics of the vessel structure is carried out depending on the at least one aggregated representation.

For example, the analysis of the hemodynamics characteristics may comprise determining a fractional flow reserve, FFR, of the vessel structure, carrying out a vessel detection of the vessel structure, carrying out a segmentation, for example a branch segmentation, of the vessel structure, carrying out a branch labeling of the vessel structure, and so forth.

According to several implementations, the algorithm for analyzing the hemodynamics characteristics comprises a trained machine learning model, MLM, which has been trained to analyze the hemodynamics characteristics using a known training technique.

In general terms, a trained MLM may mimic cognitive functions that humans associate with other human minds. In particular, by training based on training data the MLM may be able to adapt to new circumstances and to detect and extrapolate patterns. Another term for a trained MLM is “trained function”.

In general, parameters of an MLM can be adapted or updated via training. In particular, supervised training, semi-supervised training, unsupervised training, reinforcement learning and/or active learning can be used. Furthermore, representation learning, also denoted as feature learning, can be used. In particular, the parameters of the MLMs can be adapted iteratively by several steps of training. In particular, within the training a certain loss function, also denoted as cost function, can be minimized. In particular, within the training of an artificial neural network, ANN, the backpropagation algorithm can be used.

In particular, an MLM can comprise an ANN, a support vector machine, a decision tree and/or a Bayesian network, and/or the MLM can be based on k-means clustering, Q-learning, genetic algorithms and/or association rules. In particular, an ANN can be or comprise a deep neural network, a convolutional neural network or a convolutional deep neural network. Furthermore, an ANN can be an adversarial network, a deep adversarial network and/or a generative adversarial network, GAN.

According to a further aspect of one or more example embodiments of the present invention, a data processing system is provided. The data processing system is configured to carry out a computer-implemented method according to one or more example embodiments of the present invention.

In the present disclosure, the expressions “data processing system” and “at least one data processing device” may be used interchangeably. A data processing device may in particular be understood as a data processing device which comprises processing circuitry. The data processing device can therefore in particular process data to perform computing operations. This may also include operations to perform indexed accesses to a data structure, for example a look-up table, LUT, as well as a data processing process implemented in hardware.

In particular, the data processing device may include one or more computers, one or more microcontrollers, and/or one or more integrated circuits, for example, one or more application-specific integrated circuits, ASIC, one or more field-programmable gate arrays, FPGA, and/or one or more systems on a chip, SoC. The data processing device may also include one or more processors, for example one or more microprocessors, one or more central processing units, CPU, one or more graphics processing units, GPU, and/or one or more signal processors, in particular one or more digital signal processors, DSP. The data processing device may also include a physical or a virtual cluster of computers or other of said units.

In various embodiments, the data processing device includes one or more hardware and/or software interfaces and/or one or more memory units.

A memory unit may be implemented as a volatile data memory, for example a dynamic random access memory, DRAM, or a static random access memory, SRAM, or as a non-volatile data memory, for example a read-only memory, ROM, a programmable read-only memory, PROM, an erasable programmable read-only memory, EPROM, an electrically erasable programmable read-only memory, EEPROM, a flash memory or flash EEPROM, a ferroelectric random access memory, FRAM, a magnetoresistive random access memory, MRAM, or a phase-change random access memory, PCRAM.

According to a further aspect of one or more example embodiments of the present invention, a medical imaging system is provided. The medical imaging system comprises an imaging device, which is configured to generate a sequence of consecutive medical images depicting a vessel structure, for example an X-ray imaging device, in particular an X-ray angiography device and/or a C-arm device. The medical imaging system comprises a data processing system, which is configured to generate a respective graph representation of the vessel structure for each medical image of the sequence of consecutive medical images, and to carry out a computer-implemented method for generating at least one aggregated representation of the vessel structure, according to one or more example embodiments of the present invention, based on the graph representations.

Further implementations of the medical imaging system, according to one or more example embodiments of the present invention, follow directly from the various embodiments of the method and the computer-implemented method, according to one or more example embodiments of the present invention, and vice versa. In particular, individual features and corresponding explanations as well as advantages relating to the various implementations of the method and the computer-implemented method, according to one or more example embodiments of the present invention, can be transferred analogously to corresponding implementations of the medical imaging system, according to one or more example embodiments of the present invention. In particular, the medical imaging system, according to one or more example embodiments of the present invention, is designed or programmed to carry out the method or the computer-implemented method, according to one or more example embodiments of the present invention. In particular, the medical imaging system, according to one or more example embodiments of the present invention, carries out the method or the computer-implemented method, according to one or more example embodiments of the present invention.

According to a further aspect of one or more example embodiments of the present invention, a computer program comprising instructions is provided. When the instructions are executed by a data processing system, the instructions cause the data processing system to carry out a computer-implemented method, according to one or more example embodiments of the present invention.

The instructions may be provided as program code, for example. The program code can, for example, be provided as binary code or assembler and/or as source code of a programming language, for example C, and/or as program script, for example Python.

According to a further aspect of one or more example embodiments of the present invention, a further computer program comprising further instructions is provided. When the further instructions are executed by a medical imaging system, according to one or more example embodiments of the present invention, in particular by the data processing system of the medical imaging system, the instructions cause the medical imaging system to carry out a method for generating at least one aggregated representation of a vessel structure, according to one or more example embodiments of the present invention, or a method for analyzing a hemodynamics characteristics of a vessel structure, according to one or more example embodiments of the present invention.

The further instructions may be provided as program code, for example. The program code can for example be provided as binary code or assembler and/or as source code of a programming language, for example C, and/or as program script, for example Python.

According to a further aspect of one or more example embodiments of the present invention, a computer-readable storage medium storing a computer program and/or a further computer program, according to one or more example embodiments of the present invention, is provided.

The computer program, the further computer program, and the computer-readable storage medium are respective computer program products comprising the instructions and/or the further instructions.

Further features and feature combinations of the one or more example embodiments of the present invention are obtained from the figures and their description as well as the claims. In particular, further implementations of one or more example embodiments of the present invention may not necessarily contain all features of one of the claims. Further implementations of one or more example embodiments of the present invention may comprise features or combinations of features, which are not recited in the claims.

### BRIEF DESCRIPTION OF THE DRAWINGS

In the following, the present invention will be explained in detail with reference to specific exemplary implementations and respective schematic drawings. In the drawings, identical or functionally identical elements may be denoted by the same reference signs. The description of identical or functionally identical elements is not necessarily repeated with respect to different figures. In the figures,

FIG. 1 shows schematically an exemplary implementation of a medical imaging system according to the present invention;

FIG. 2 shows schematically a medical image depicting a vessel structure and a corresponding graph representation of the vessel structure;

FIG. 3 shows a schematic flow diagram of an exemplary implementation of a computer-implemented method for generating at least one aggregated representation of a vessel structure according to the present invention;

FIG. 4 shows a schematic flow diagram of a further exemplary implementation of a computer-implemented method for generating at least one aggregated representation of a vessel structure according to the present invention;

FIG. 5 shows a schematic flow diagram of a further exemplary implementation of a computer-implemented method for generating at least one aggregated representation of a vessel structure according to the present invention; and

FIG. 6 shows schematically an artificial neural network.

### DETAILED DESCRIPTION

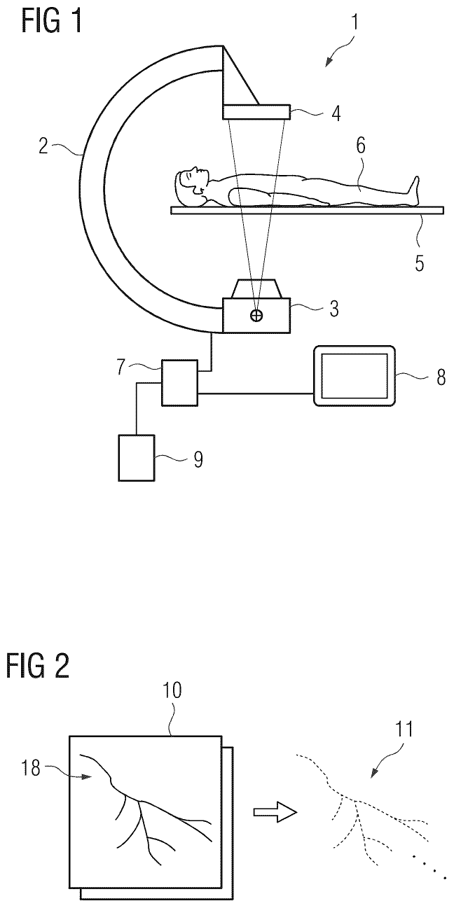

FIG. 1 shows schematically an exemplary implementation of a medical imaging system 1 according to the present invention. The medical imaging system comprises an imaging device, for example an X-ray imaging device.

The X-ray imaging device comprises a source unit 3 with an X-ray source, a detector unit 4 with an X-ray detector, and a control system 7, which is configured to control the X-ray source and the X-ray detector to generate X-ray images 10 depicting a vessel structure 18, for example a coronary vessel structure, of a patient 6, as depicted schematically in FIG. 2. The X-ray imaging device may, for example, comprise a patient table 5, on which the patient 6 is arranged.

The medical imaging system 1 comprises a data processing system 9, according to one or more example embodiments of the present invention, which is adapted to carry out a computer-implemented method for generating at least one aggregated representation of the vessel structure 18, according to one or more example embodiments of the present invention. In the following, several functions and method steps are described to be carried out by the control system 7, while other functions and method steps are described to be carried out by the data processing system 9. It is noted that the functions and method steps may also be distributed in different ways in alternative implementations.

For example, the control system 7 may adjust various imaging parameters of the X-ray imaging device including, for example, exposure parameters like a peak kilovoltage of the X-ray source, a tube current of the X-ray source, and/or an X-ray pulse duration. For example, the control system 7 may adjust further imaging parameters like a filter material and/or filter thickness of an X-ray filter, for example a copper filter, by placing the appropriate X-ray filter into the beam path or by removing it from the beam path, respectively. For example, the control system 7 may adjust further imaging parameters like a collimator opening size of an X-ray collimator. For example, the control system 7 may bring the X-ray collimator into the beam path or remove it from the beam path, respectively. For example, the control system 7 may adjust further imaging parameters like a gain factor of the X-ray detector. For example, the control system 7 may bring an anti-scattering grid into the beam path or remove it from the beam path, respectively.

In some implementations, the X-ray imaging device comprises a display device 8, wherein the control system 7 is configured to control the display device 8 to display the X-ray images 10 or processed X-ray images.

In some implementations, the X-ray imaging device is designed as a C-arm fluoroscopy system, wherein the source unit 3 and the detector unit 4 are attached opposite to each other on a C-arm 2, which can be rotated around different axes. The corresponding motions are denoted as angular and orbital motion respectively. In some implementations, apart from the rotational motion of the C-arm 2, the patient table 5 and the C-arm 2 may be positioned relative to each other by respective translatory motions of the C-arm 2 and/or the patient table 5. Consequently, the position and/or orientation of the X-ray source with respect to the object 6 and the position and/or orientation of the X-ray detector with respect to the object 6 may be adjusted exactly to the required imaging perspective.

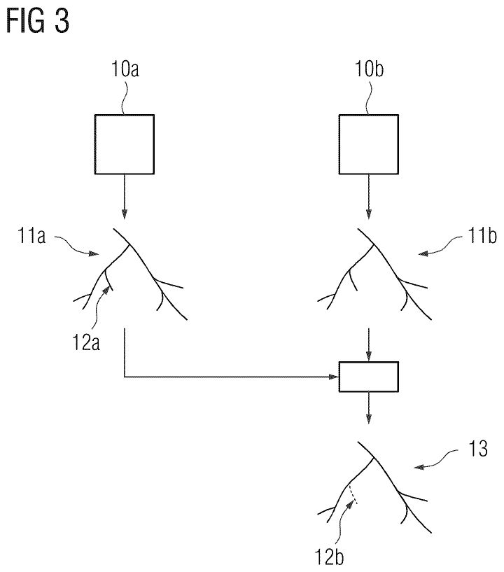

FIG. 3 shows a schematic flow diagram of an exemplary implementation of a computer-implemented method for generating at least one aggregated representation of a vessel structure 18 according to the present invention. Therein, the data processing system 9 receives a sequence of consecutive medical images 10, 10a, 10b depicting the vessel structure 18 from the medical imaging device and generates graph representations 11, 11a, 11b, in particular rooted graph representations 11, of the vessel structure 18, each of the graph representations 11, 11a, 11b corresponding to one of the medical images 10, 10a, 10b of. Each of the graph representations 11, 11a, 11b comprises a plurality of nodes and respective spatial coordinates for each node, wherein the plurality of nodes is arranged according to at least one segment 12a of the respective graph representation 11, 11a, 11b.

For each segment 12a of a first graph representation 11a of the plurality of graph representations 11, 11a, 11b, it is determined whether a second graph representation 11b of the plurality of graph representations 11, 11a, 11b comprises a segment corresponding to the respective segment 12a of the first graph representation 11, 11a, 11b. If it is found that the second graph representation 11b does not comprise a segment corresponding to the respective segment 12a of the first graph representation 11a, the second graph representation 11b is augmented by adding an additional segment 12b corresponding to the respective segment 12a of the first graph representation 11a to the second graph representation 11b. The at least one aggregated representation is generated depending on the augmented second graph representation 13.

In the example of FIG. 3, the first graph representation 11a comprises exactly one segment 12a, which does not have a respective counterpart in the second graph representation 11b. Thus, the augmented second graph representation 13 corresponds to the second graph representation 11b with the additional segment 12b. In general, however, it is also possible that there is no such additional segment 12b to be added or there is more than one additional segment 12b to be added.

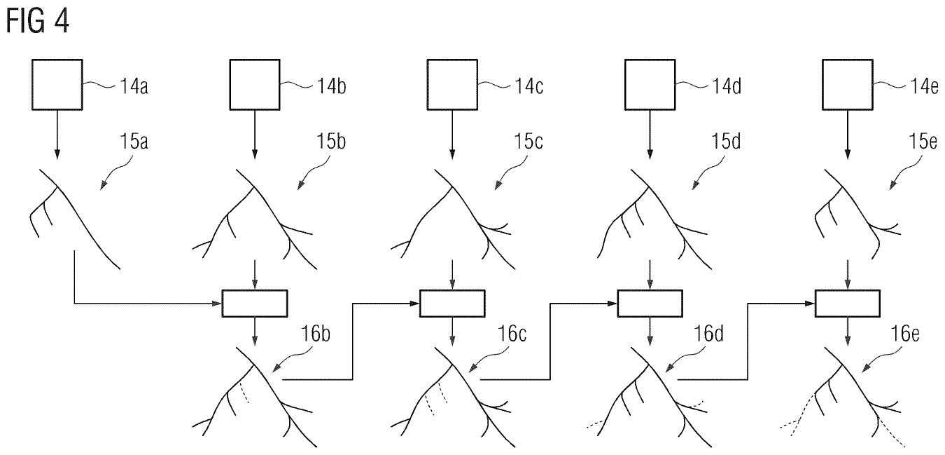

The total number of the medical images 10 may be greater than two and, consequently, the number of graph representations 11 to be aggregated may be greater than two as well. As an example, FIG. 4 depicts schematically a scenario with five medical images 14a, 14b, 14c, 14d, 14e and five corresponding graph representations 15a, 15b, 15c, 15d, 15e extracted from the medical images 14a, 14b, 14c, 14d, 14e.

In a further exemplary implementation of the computer-implemented method according to the present invention, the aggregation is carried out as described in the following. First, the graph representation 15b is compared to the preceding graph representation 15a. An augmented graph representation 16b is generated by adding respective segments of the graph representation 15a, which do not have a corresponding counterpart in the graph representation 15b, to the graph representation 15b. Then, the graph representation 15c is compared to the augmented graph representation 16b. An augmented graph representation 16c is generated by adding respective segments of the augmented graph representation 16b, which do not have a corresponding counterpart in the graph representation 15c, to the graph representation 15c.

Then, the graph representation 15d is compared to the augmented graph representation 16c. An augmented graph representation 16d is generated by adding respective segments of the augmented graph representation 16c, which do not have a corresponding counterpart in the graph representation 15d, to the graph representation 15d. Finally, the graph representation 15e is compared to the augmented graph representation 16d. An augmented graph representation 16e is generated by adding respective segments of the augmented graph representation 16d, which do not have a corresponding counterpart in the graph representation 15e, to the graph representation 15e. Consequently, the resulting augmented graph representation 16e comprises all segments of all of the graph representations 15a, 15b, 15c, 15d, 15e.

The augmented graph representation 16e may be a result of the computer-implemented method, for example in a scenario where the aggregation is carried out online or live, right after the respective medical image 14a, 14b, 14c, 14d, 14e has been generated. Once another medical image is generated, the next aggregation step may be carried out analogously.

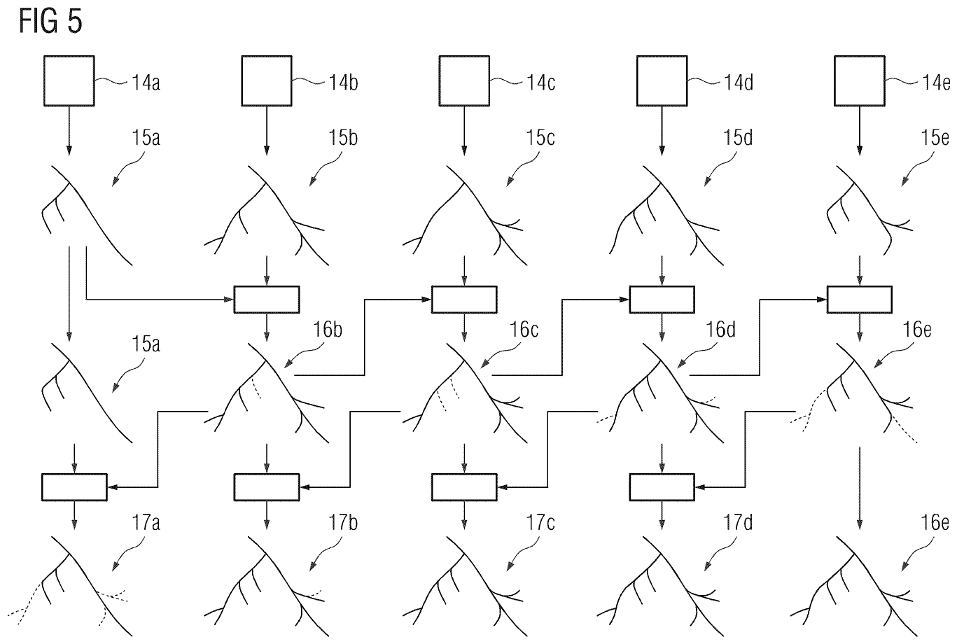

Alternatively, the medical images 14a, 14b, 14c, 14d, 14e may have been generated previously and afterwards the aggregation is carried out. A corresponding further exemplary implementation of the computer-implemented method according to the present invention is shown schematically in FIG. 5.

The steps as explained with respect to FIG. 4 are carried out also in this implementation, they may be denoted as forward processing. Afterwards, the augmented graph representations 16b, 16c, 16d, 16e are processed in the opposite order, which may be denoted as backwards processing.

In particular, the augmented graph representation 16d is compared to the augmented graph representation 16e. An augmented graph representation 17d is generated by adding respective segments of the augmented graph representation 16e, which do not have a corresponding counterpart in the augmented graph representation 16d, to the augmented graph representation 16d. Then, the augmented graph representation 16c is compared to the augmented graph representation 16d. An augmented graph representation 17c is generated by adding respective segments of the augmented graph representation 16d, which do not have a corresponding counterpart in the augmented graph representation 16c, to the augmented graph representation 16c. Then, the augmented graph representation 16b is compared to the augmented graph representation 16c. An augmented graph representation 17b is generated by adding respective segments of the augmented graph representation 16c, which do not have a corresponding counterpart in the augmented graph representation 16b, to the augmented graph representation 16b. Finally, the graph representation 15a is compared to the augmented graph representation 16b. An augmented graph representation 17a is generated by adding respective segments of the augmented graph representation 16b, which do not have a corresponding counterpart in the graph representation 15a, to the graph representation 15a.

The resulting augmented graph representations 17a, 17b, 17c, 17d, 16e each comprise all segments of all of the graph representations 15a, 15b, 15c, 15d, 15e.

In some implementations, during the forward and backward processing, a method of mapping the locations of all nodes pertaining to a graph representation 15a, 15b, 15c, 15d, 15e in a given frame onto corresponding locations in another frame is employed, which will provide more accurate results. This can be accomplished, for example, by applying pre-computed deformation fields that take into account the temporal movement of the vessel structure 18, for example due to cardiac motion, respiratory motion, table panning etc. over time. These deformation fields can be computed based on the angiographic data and/or based on additional information previously extracted, for example vesselness heatmaps, either via a standard deformable registration algorithm, or by applying an MLM, for example a deep neural network, trained specifically for this task. Thus, merging segments pertaining to graph representation 15a, 15b, 15c, 15d, 15e extracted from two different frames employs these deformation fields to bring the node locations in the same coordinate space, then a fuzzy check may be used to determine whether that segment or some portion of it is already covered or not, for example via calculating the distances of the segment's nodes to the closest respective node and applying thresholding or similar techniques.

In other implementations, instead of performing the aggregation sequentially across frames as described with regard to FIG. 4 and FIG. 5, a divide-and-conquer approach may be employed. Such approaches process the graph representations 15a, 15b, 15c, 15d, 15e in a similar fashion to a merge-sort algorithm, by dividing the sequence of graph representations 15a, 15b, 15c, 15d, 15e into subsequences until the trivial subsequence containing two graph representations 15a, 15b, 15c, 15d, 15e is reached, then merging the graph representations 15a, 15b, 15c, 15d, 15e in a bottom-up fashion.

In some implementations, a post-processing step ensuring segment ordering consistency may be performed by adjusting the order of child nodes for the multifurcation nodes such that a tree traversal algorithm, for example a depth first traversal algorithm, applied on any two trees yields pairs of corresponding segments.

In some implementations, segments for which there is not enough supporting evidence, for example a segment being detected only in a small subset of frames, or a segment having a different trace in a subset of frames due to overlaps, may be removed from the aggregated tree model or marked as uncertain.

In several implementations of the present invention, a task is to aggregate graph representations 11 of a vessel structure 18, extracted from multiple frames into a unified aggregated graph representation, which is designed to robustly aggregate topological information of the vessel structure into a frame-agnostic topology model. This allows establishing correspondences to match specific portions of the vessel structure 18, for example side branches, landmarks, bifurcation points, and so forth, across frames. Accurate matching at the topology level is very helpful for subsequent analysis tasks involving multiple frames.

In several implementations of the present invention, a tree aggregation method is performed, which is tailored towards robust aggregation of noisy coronary topology graph representations 11, which were individually extracted from multiple frames of the same coronary angiography sequence, that is the same underlying anatomy and the same patient 6, but at different time points and/or different heart phases and/or different image quality and hence different quality of the extracted graph representations 11. The graph representations 11 may also implicitly hold an order of branches or segments or more generally, subsets of nodes in the graph representations 11, which can be used to uniquely identify a subset of nodes in the graph representation 11, for example using a numeric identifier. This ordering may represent, for example, the hemodynamic significance of each segment as determined based on geometric attributes such as length and radius.

It is noted, however, that the method for extracting the graph representations 11 from the medical images 10 is not necessarily required to generate a consistent ordering across multiple frames. Furthermore, there may be inconsistencies, for example in the number of extracted segments and in their ordering.

For example, a trained MLM, for example a trained artificial neural network, ANN, may be applied to the at least one aggregated representation to analyze a hemodynamics characteristics of the vessel structure 18, for example to compute an FFR.

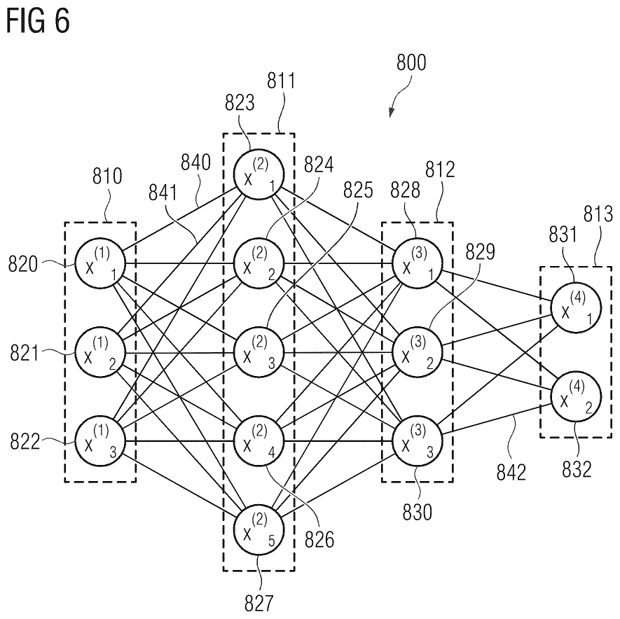

FIG. 6 displays an embodiment of an ANN 800, which is for example designed as a multi-layer perceptron, MLP. The ANN 800 comprises nodes 820, 821, 822, 823, 824, 825, 826, 827, 828, 829, 830, 832 and edges 840, 841, 842, wherein each edge 840, . . . , 842 is a directed connection from a first node 820, . . . , 832 to a second node 820, . . . , 832. In general, the first node 820, . . . , 832 and the second node 820, . . . , 832 are different nodes 820, . . . , 832. It is, however, also possible that the first node 820, . . . , 832 and the second node 820, . . . , 832 are identical. For example, in FIG. 6, the edge 840 is a directed connection from the node 820 to the node 823, and the edge 842 is a directed connection from the node 830 to the node 832. An edge 840, . . . , 842 from a first node 820, . . . , 832 to a second node 820, . . . , 832 is also denoted as ingoing edge for the second node 820, . . . , 832 and as outgoing edge for the first node 820, . . . , 832.

In this example, the nodes 820, . . . , 832 of the artificial neural network 800 can be arranged in layers 810, . . . , 813, wherein the layers can comprise an intrinsic order introduced by the edges 840, . . . , 842 between the nodes 820, . . . , 832. In particular, edges 840, . . . , 842 can exist only between neighboring layers of nodes. In the displayed example, there is an input layer 810 comprising only nodes 820, . . . , 822 without an incoming edge, an output layer 813 comprising only nodes 831, 832 without outgoing edges, and hidden layers 811, 812 in-between the input layer 810 and the output layer 813. In general, the number of hidden layers 811, 812 can be chosen arbitrarily. In an MLP, this number is at least one. The number of nodes 820, . . . , 822 within the input layer 810 usually relates to the number of input values of the artificial neural network 800, and the number of nodes 831, 832 within the output layer 813 usually relates to the number of output values of the artificial neural network 800.

In particular, a real number can be assigned as a value to every node 820, . . . , 832 of the artificial neural network 800. Here, x(n)i denotes the value of the i-th node 820, . . . , 832 of the n-th layer 810, . . . , 813. The values of the nodes 820, . . . , 822 of the input layer 810 are equivalent to the input values of the artificial neural network 800. The values of the nodes 831, 832 of the output layer 813 are equivalent to the output value of the artificial neural network 800. Furthermore, each edge 840, . . . , 842 can comprise a weight being a real number. In particular, the weight is a real number within the interval [−1, 1] or within the interval [0, 1]. Here, w(m,n)i,j denotes the weight of the edge between the i-th node 820, . . . , 832 of the m-th layer 810, . . . , 813 and the j-th node 820, . . . , 832 of the n-th layer 810, . . . , 813. Furthermore, the abbreviation w(n)i,j is defined for the weight w(n,n+1)i,j. In particular, to calculate the output values of the neural network 800, the input values are propagated through the neural network 800. In particular, the values of the nodes 820, . . . , 832 of the (n+1)-th layer 810, . . . , 813 can be calculated based on the values of the nodes 820, . . . , 832 of the n-th layer 810, . . . , 813 by

$${x}_{j}^{( n + 1 )} = f ( {\sum }_{i} {x}_{i}^{( n )} {w}_{i , j}^{( n )} ) .$$

Herein, the function f is denoted as transfer function or activation function. Known transfer functions are step functions, the sigmoid functions, for example the logistic function, the generalized logistic function, the hyperbolic tangent, the arctangent function, the error function, the smoothstep function, or rectifier functions. The transfer function is, for example, used for normalization purposes. In particular, the values are propagated layer-wise through the neural network 800, wherein values of the input layer 810 are given by the input of the neural network 800, wherein values of the first hidden layer 811 can be calculated based on the values of the input layer 810 of the neural network 800, wherein values of the second hidden layer 812 can be calculated based on the values of the first hidden layer 811, and so forth.

In order to set the values w(m,n)i,j for the edges, the neural network 800 has to be trained using training data. In particular, training data comprises training input data and training output data (denoted as ti). For a training step, the neural network 800 is applied to the training input data to generate calculated output data. In particular, the training data and the calculated output data comprise a number of values, said number being equal with the number of nodes of the output layer. In particular, a comparison between the calculated output data and the training data is used to recursively adapt the weights within the neural network 800 (backpropagation algorithm). In particular, the weights are changed according to

$${w}_{i , j}^{′ ( n )} = {w}_{i , j}^{( n )} - γ {δ}_{j}^{( n )} {x}_{i}^{( n )} ,$$

wherein γ is a predefined learning rate, and the numbers δ(n)j can be recursively calculated as

$${δ}_{j}^{( n )} = ( {\sum }_{k} {δ}_{k}^{( n + 1 )} {w}_{j , k}^{( n + 1 )} ) {f}^{′} ( {x}_{i}^{( n )} {w}_{i , j}^{( n )} )$$

based on δ(n+1)j, if the (n+1)-th layer is not the output layer 813, and

$${δ}_{j}^{( n )} = ( {x}_{j}^{( n + 1 )} - {t}_{j}^{( n + 1 )} ) {f}^{′} ( {x}_{i}^{( n )} {w}_{i , j}^{( n )} ) ,$$

if the (n+1)-th layer is the output layer 813, wherein f′ is the first derivative of the activation function, and t(n+1)j is the comparison training value for the j-th node of the output layer 813.

Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A computer-implemented method for generating at least one aggregated representation of a vessel structure, the computer-implemented method comprising:
receiving graph representations of the vessel structure, each of the graph representations corresponding to a respective medical image of a sequence of consecutive medical images depicting the vessel structure, wherein
each respective graph representation includes a plurality of nodes and respective spatial coordinates for each node, and
the plurality of nodes are arranged according to at least one segment of the respective graph representation;
for each respective segment of a first graph representation of the graph representations,
determining whether a second graph representation of the graph representations includes the segment corresponding to the respective segment of the first graph representation, and
in response to determining that the second graph representation does not include a segment corresponding to the respective segment of the first graph representation, augmenting the second graph representation by adding, to the second graph representation, an additional segment corresponding to the respective segment of the first graph representation; and
generating the at least one aggregated representation depending on the augmented second graph representation.

2. The computer-implemented method according to claim 1, further comprising:
ordering the graph representations into a single ordered sequence of graph representations;
for each iteration of N−1 iterations, indexing the graph representations by i∈[1,N]∩N, wherein a total number of graph representations of the single ordered sequence is N, and wherein the N−1 iterations start with i=1 and end with i=N−1; and
for each respective segment of an i-th graph representation of the single ordered sequence
determining whether an (i+1)-th graph representation of the single ordered sequence includes a segment corresponding to the respective segment of the i-th graph representation of the single ordered sequence, and
in response to determining that the (i+1)-th graph representation does not include the segment corresponding to the respective segment of the i-th graph representation, augmenting the (i+1)-th graph representation by adding, to the (i+1)-th graph representation, an additional segment corresponding to the respective segment of the i-th graph representation; and wherein
the at least one aggregated representation is generated depending on an augmented N-th graph representation of the single ordered sequence.

3. The computer-implemented method according to claim 2, wherein after the N−1 iterations have been carried out, the computer-implemented method further comprises:
for each iteration of N−1 further iterations, indexing the graph representations by j∈[1,N]∩N, wherein the N−1 iterations start with j=1 and end with j=N−1; and
for each respective segment of an (N−j+1)-th graph representation of the single ordered sequence
determining whether an (N−j)-th graph representation of the single ordered sequence includes a segment corresponding to the respective segment of the (N−j+1)-th graph representation of the single ordered sequence;
in response to determining that the (N−j)-th graph representation does not include the segment corresponding to the respective segment of the (N−j+1)-th graph representation, augmenting the (N−j)-th graph representation by adding, to the (N−j)-th graph representation, an additional segment corresponding to the respective segment of the (N−j+1)-th graph representation; and wherein
the at least one aggregated representation is generated depending on an augmented initial graph representation of the single ordered sequence.

4. The computer-implemented method according to claim 1, wherein
the graph representations are grouped into at least two ordered sequences of graph representations, the at least two ordered sequences including a first ordered sequence and a second ordered sequence;
for each iteration of N1−1 iterations, wherein a total number of graph representations of the first ordered sequence is N1, indexing the graph representations of the first ordered sequence by i∈[1,N1]∩N, wherein the N1−1 iterations start with i=1 and end with i=N1−1; and
for each respective segment of an i-th graph representation of the first ordered sequence
determining whether an (i+i)-th graph representation of the first ordered sequence includes a segment corresponding to the respective segment of the i-th graph representation of the first ordered sequence, and
in response to determining that the (i+i)-th graph representation of the first ordered sequence does not include the segment corresponding to the respective segment of the i-th graph representation of the first ordered sequence, augmenting the (i+i)-th graph representation of the first ordered sequence by adding, to the (i+i)-th graph representation of the first ordered sequence, an additional segment corresponding to the respective segment of the i-th graph representation of the first ordered sequence; and
wherein the at least one aggregated representation is generated depending on an augmented N1-th graph representation of the first ordered sequence.

5. The computer-implemented method according to claim 4, further comprising:
for each iteration of N2−1 further iterations, indexing the graph representations of the second ordered sequence by i∈[1,N2]∩N, wherein a total number of graph representations of the second ordered sequence is N2, and the N2−1 further iterations start with i=1 and end with i=N2−1;
for each respective segment of the i-th graph representation of the second ordered sequence,
determining whether an (i+1)-th graph representation of the second ordered sequence includes a segment corresponding to the respective segment of the i-th graph representation of the second ordered sequence, and
in response to determining that the (i+1)-th graph representation of the second ordered sequence does not include a segment corresponding to the respective segment of the i-th graph representation of the second ordered sequence, augmenting the (i+1)-th graph representation of the second ordered sequence by adding, to the (i+1)-th graph representation of the second ordered sequence, an additional segment corresponding to the respective segment of the i-th graph representation of the second ordered sequence; and wherein
the at least one aggregated representation is generated depending on an augmented N2-th graph representation of the second ordered sequence.

6. The computer-implemented method according to claim 5, wherein for each respective segment of an N1-th graph representation of the first ordered sequence, the computer-implemented method further comprises:
determining whether an N2-th graph representation of the second ordered sequence includes a segment corresponding to the respective segment of the N1-th graph representation of the first ordered sequence;
in response to determining that the N2-th graph representation of the second ordered sequence does not include a segment corresponding to the respective segment of the N1-th graph representation of the first ordered sequence, augmenting the N2-th graph representation of the second ordered sequence by adding, to the N2-th graph representation of the second ordered sequence, an additional segment corresponding to the respective segment of the N1-th graph representation of the first ordered sequence; and wherein
the at least one aggregated representation is generated depending on the augmented N2-th graph representation of the second ordered sequence.

7. The computer-implemented method according to claim 5, wherein the N1−1 iterations and the N2−1 further iterations are at least partially carried out in parallel.

8. The computer-implemented method according to claim 1, further comprising:
registering the first graph representation to the second graph representation;
computing a distance metric between a registered respective segment of the first graph representation and the second graph representation; and
determining whether the second graph representation includes a segment corresponding to the registered respective segment of the first graph representation depending on the distance metric.

9. The computer-implemented method according to claim 1, further comprising:
determining a deformation field between the first graph representation and the second graph representation; and wherein
the second graph representation is augmented by adding the additional segment to the second graph representation depending on the deformation field.

10. The computer-implemented method according to claim 1, wherein each of the graph representations is given by a graph representation.

11. A method for generating at least one aggregated representation of a vessel structure, the method comprising:
generating, by an imaging device, a sequence of consecutive medical images depicting the vessel structure;
generating graph representations of the vessel structure for the sequence of consecutive medical images; and
performing the computer-implemented method according to claim 1 based on the graph representations.

12. The method according to claim 11, wherein the sequence of consecutive medical images is generated as a sequence of consecutive two-dimensional images.

13. A data processing system configured to carry out the computer-implemented method according to claim 1.

14. A medical imaging system comprising:
an imaging device configured to generate a sequence of consecutive medical images depicting a vessel structure; and
a data processing system configured to
generate graph representations of the vessel structure for the sequence of consecutive medical images, and
perform the computer-implemented method according to claim 1 based on the graph representations.

15. A non-transitory computer-readable storage medium storing computer-executable instructions that, when executed by a data processing system, cause the data processing system to perform the computer-implemented method according to claim 1.

16. The computer-implemented method according to claim 6, wherein the N1−1 iterations and the N2−1 further iterations are at least partially carried out in parallel.

17. The computer-implemented method according to claim 2, further comprising:
registering the first graph representation to the second graph representation;
computing a distance metric between a registered respective segment of the first graph representation and the second graph representation; and
determining whether the second graph representation includes a segment corresponding to the registered respective segment of the first graph representation depending on the distance metric.

18. The computer-implemented method according to claim 3, further comprising:
registering the first graph representation to the second graph representation;
computing a distance metric between a registered respective segment of the first graph representation and the second graph representation; and
determining whether the second graph representation includes a segment corresponding to the registered respective segment of the first graph representation depending on the distance metric.

19. The computer-implemented method according to claim 2, further comprising:
determining a deformation field between the first graph representation and the second graph representation; and wherein
the second graph representation is augmented by adding the additional segment to the second graph representation depending on the deformation field.

20. The computer-implemented method according to claim 3, further comprising:
determining a deformation field between the first graph representation and the second graph representation; and wherein
the second graph representation is augmented by adding the additional segment to the second graph representation depending on the deformation field.
